(19) United States
(12) Patent Application Publication
(10) Pub. No.: US 2022/0022746 A1
(43) Pub. Date: Jan. 27, 2022

(54) THERMO-BOARDING PASS SYSTEM

(71) Applicant: CUSI Aerospace LLC, Dripping Springs, TX (US)

(72) Inventors: Kempen CLOETE, Johnson City, TX (US); Erich HOPKINS, Millsboro, DE (US); James Nicholas OLMSTED, Millsboro, DE (US); Michael Henry BORFITZ, Seattle, WA (US)

(21) Appl. No.: 16/935,905

(22) Filed: Jul. 22, 2020

(51) Int. Cl. A61B 5/00; G07C 9/37

(52) U.S. Cl. CPC A61B 5/0008; G07C 9/37; A61B 5/6888

(57) ABSTRACT

A method and system of screening persons boarding an aircraft to detect a febrile condition of each person are disclosed. According to one aspect, a method includes: detecting an open condition of a door of the aircraft; when the door is in an open condition, detecting a presence of a person entering through the door; thermally and optically imaging the person; determining a febrile condition of the person; and when the person is in a febrile condition, alerting aircraft employees.

### FIELD

The present technology is generally related to aircraft passenger screening and more particularly, to thermal screening of passengers as they board the aircraft.

### BACKGROUND

Air Travel has a perception of being perilous for the airline passenger. Passengers know that contagious diseases may be spread by traveling in close proximity to other passengers in a confined area for any amount of time.

### SUMMARY

The techniques of this disclosure generally relate to aircraft passenger screening and more particularly, to thermal screening of passengers as they board the aircraft.

The Thermo-Boarding Pass System (TBPS) is a solution to actively monitor international flights as well as domestic flights. In some embodiments, the TBPS actively processes passengers as they are boarding the aircraft, just before that passenger stows carry-on bags and takes their seat. In addition, the TBPS also may be used to scan all flight crew, maintenance and service personnel and/or anyone else that steps through a doorway onto the aircraft.

The TBPS is a non-invasive method for actively monitoring for above normal temperatures of airline travelers. The TBPS is designed be installed in the aircraft's main-deck overhead ceiling panels located at each doorway entry. The TBPS provides a display located within a crew member's control. In today's traveling environment, the TBPS provides the alerting necessary to provide a level of safer travel and some assurance for the passenger of a better environment from contagious diseases such as influenza, H1N1, Avian flu (bird flu) SARS, MERS, Ebola and COVID-19.

In some embodiments, the TBPS is used to process passengers for commercial airline travel, aircraft charter services, and track passengers, aircraft maintenance personnel, aircraft service personnel, and aircraft flight crew, pilots and flight attendants.

To accurately track people for the purpose of maintaining health and security, note that humans are able to by-pass normal ground security protocols prior to entering any aircraft. In addition, security protocols must recognize that employers are not necessarily aware nor are they required to exactly know who has entered an unsecured and non-monitored aircraft.

The TBPS enables continuous on-board aircraft monitoring of the aircraft as long as an aircraft door is opened. The Thermo-boarding Pass System may be installed at each doorway to monitor who enters through the doorway.

The Thermo-boarding Pass System may be manufactured with the following characteristics: the ability to count, monitor, identify, and record identifying human facial features, and store all imaged data; receive and transmit data; power on and off automatically; display data and images; thermally scan heat signatures for onboard analysis of identifying febrile humans; and provide an image of written identification.

Stored imaging data collected by the TBPS may be processed on board by flight crew through examination of the stored data using an access method such as with a computing device designed to process software and images for viewing by the crew. Stored data may also be transmitted to a larger database for further processes of contact tracing, security screening for security violations, and metrics for timely boarding, and weight. The stored data may also be transmitted in whole or in part to a mobile device such as a cell phone, and an application program stored on the mobile device may query the TBPS. The stored data may be communicated to the flight crew, the flight attendants, ground personnel, security personnel or other personnel Transmission paths that may be used to communicate information from and to the TBPS include cellular while on ground, satellite, air-to-ground phone, ground Wi-Fi 802.11 while connected to a ground network, as determined by other equipment on the aircraft. Also, data may be downloaded to a disc or to a thumb drive.

According to one aspect, a method is provided for thermal screening of persons. The method includes detecting an open condition of a door of the aircraft. When the door is in an open condition, the method includes detecting a presence of a person entering through the door. The method also includes thermally and optically imaging the person, determining a febrile condition of the person; and when the person is in a febrile condition, generating an alert. An alert can be a signal, an audible tone, an audible voice message, an email, a text, a flashing light, a visual message on a video display, and/or a phone call, for example.

According to this aspect, in some embodiments, the method includes storing the determination of febrile condition in a database for comparison to subsequent and prior determinations of febrile condition. In some embodiments, the method includes determining an identity of a person in a febrile condition based at least in part on facial recognition of the person. In some embodiments, the method includes storing the identity of the person in a database and correlating the identity of the person with the febrile condition of the person. In some embodiments, the method includes displaying on a video display the febrile condition and identity of the person. In some embodiments, the method includes thermal imaging by a forward looking infrared sensor mounted in a ceiling of the aircraft in proximity of the door. In some embodiments, the method includes storing in a first database a febrile condition of an aircraft employee. In some embodiments, the method includes recording a count of persons entering the aircraft that have a febrile condition or that have a non-febrile condition. In some embodiments, the method includes storing thermal imaging data in a database. In some embodiments, the method includes storing contact tracing information for a person determined to be in a febrile condition.

According to another aspect, a thermo-boarding pass system (TBPS) for installation on an aircraft is provided with processing circuitry configured to detect an open condition of a door of the aircraft. When the door is in an open condition, the processing circuitry is configured to: detect a presence of a person entering through the door; thermally and optically image the person; determine a febrile condition of the person; and when the person is in a febrile condition, alert aircraft employees.

According to this aspect, in some embodiments, the processing circuitry is further configured to store the determination of febrile condition in a database for comparison to subsequent and prior determinations of febrile condition. In some embodiments, the processing circuitry is further configured to determine an identity of a person in a febrile condition based on facial recognition of the person. In some embodiments, the processing circuitry is further configured to store the identity of the person in a database and correlate the identity of the person with the febrile condition of the person. In some embodiments, the system is further configured to comply with at least one Federal Aviation Administration (FAA) requirement that includes at least one of an electrical loading requirement, a safe wiring requirement, a thermal overload limit, and a constraint on electromagnetic interference. In some embodiments, the processing circuitry is further configured to process a thermal image captured by a forward looking infrared sensor mounted in a ceiling of the aircraft in proximity of the door. In some embodiments, the processing circuitry is further configured to store in a first database a febrile condition of an aircraft employee. In some embodiments, the processing circuitry is further configured to record a count of persons entering the aircraft that have a febrile condition or that have a non-febrile condition. In some embodiments, the processing circuitry is further configured to store thermal imaging data in a database. In some embodiments, the processing circuitry is further configured to provide storing contact tracing information for a person determined to be in a febrile condition.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an illustration of a forward portion of an aircraft showing a location of thermo-boarding pass system;

FIG. 2 is an illustration showing a location of a thermo-boarding pass system;

FIG. 3 is an illustration showing the thermo-boarding pass system mounted in a ceiling of the aircraft;

FIG. 4 is an illustration of the ceiling mounted thermal sensing system of the TBPS;

FIG. 5 is an illustration of the thermal sensing system showing a lens;

FIG. 6 is a block diagram of a thermo-boarding pass system according to principles set forth herein; and

FIG. 7 is a flowchart of an example process for thermal screening of persons on an aircraft.

### DETAILED DESCRIPTION

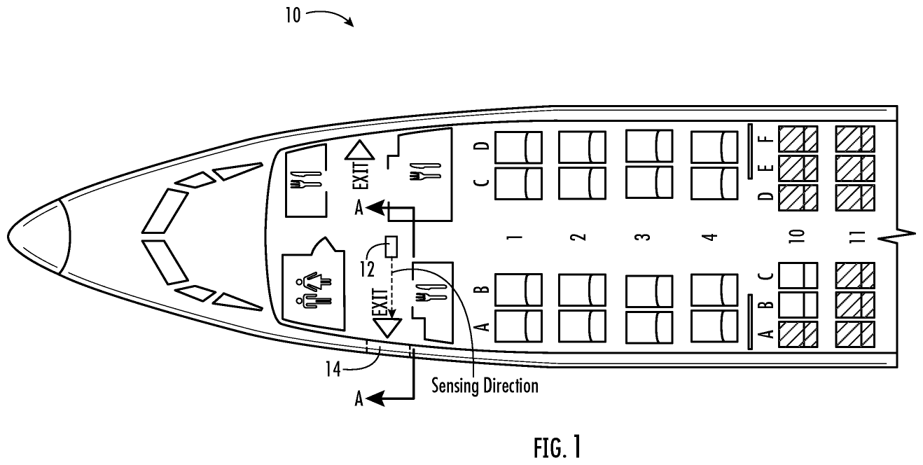

The present technology is generally related to aircraft passenger screening and more particularly, to thermal screening of passengers as they board the aircraft. FIG. 1 is an illustration of a forward portion of an aircraft 10 showing a location of thermo-boarding pass system 12 or at least part thereof, mounted in a ceiling of the aircraft just inside the door 14 of the aircraft 10. In some embodiments, there is a TBPS 12 at each door 14 of the aircraft 10.

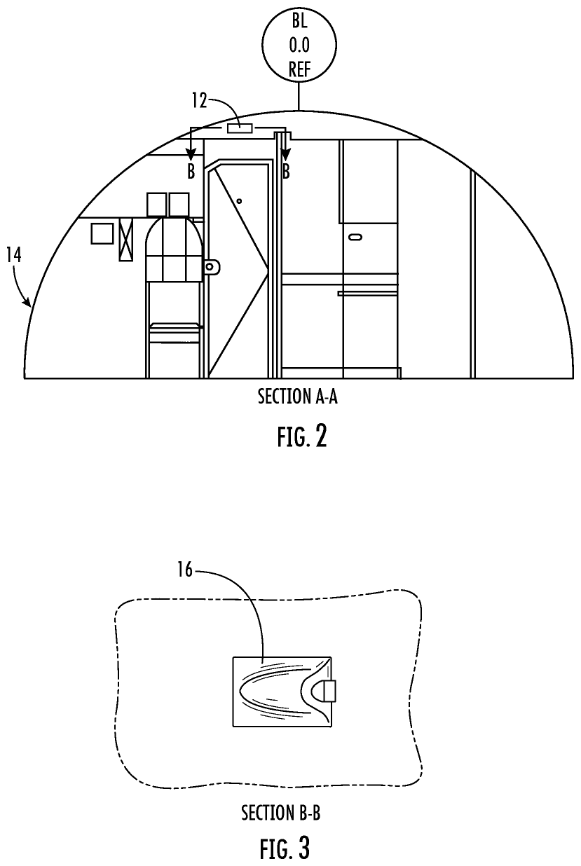

FIG. 2 is an illustration showing a location of a thermo-boarding pass system 12 when viewing the interior of the aircraft 10 facing forward. FIG. 3 is an illustration showing the thermo-boarding pass system 12 mounted in a ceiling of the aircraft 10.

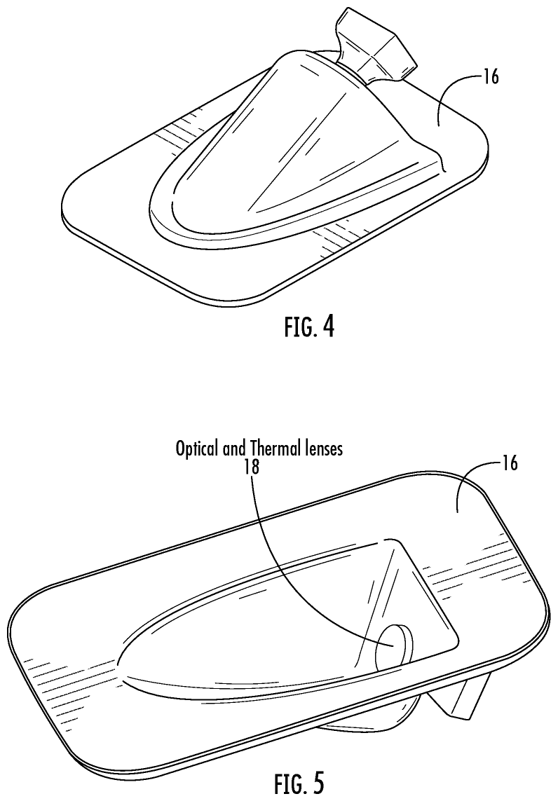

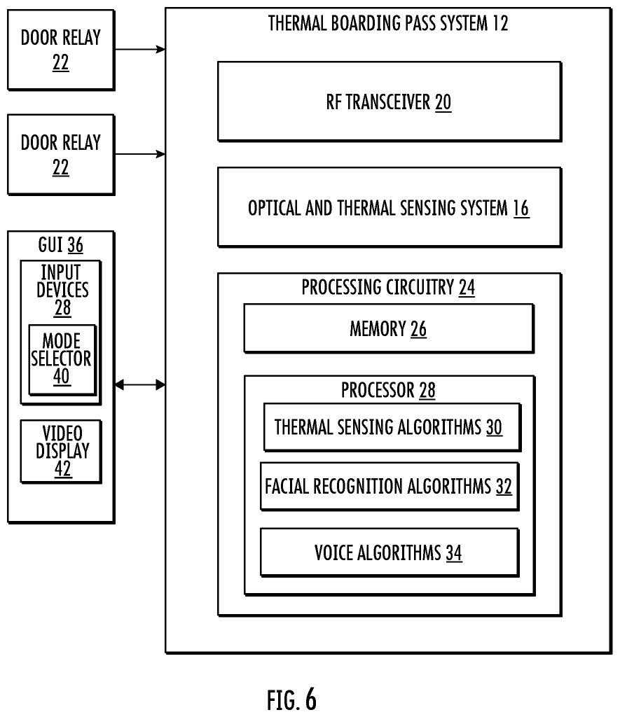

FIG. 4 is an illustration of a top view of a ceiling mounted optical and thermal sensing system 16 of the TBPS 12. FIG. 5 is an illustration of a bottom view of the optical and thermal sensing system 16 showing a at least one lens 18 for focusing the thermal energy detected by the optical and thermal sensing system 16 and to focus an image for optical facial recognition.

FIG. 4 is a block diagram of a thermo-boarding pass system 12 according to principles set forth herein. The TBPS 12 may include an RF transceiver 20 configured to enable the TBPS 12 to communicate with other units of the aircraft 10, such as a door relay 22 which is open or closed according to whether the door 14 is open or closed. Thus, in some embodiments, the TBPS 12 is activated when the door 14 is open and deactivated when the door 14 is closed. The optical and thermal sensing system 16 may comprise a forward looking infrared detector capable of detecting heat energy emanating from a passenger. Note that although the optical and thermal sensing system 16 is shown within the TBPS unit 12, in some embodiments, the optical and thermal sensing system 16 is mounted in the ceiling of the aircraft 10, separated physically but connected electrically or wirelessly to the TBPS unit 12. Note that the TBPS 12 may be battery powered and/or powered by the aircraft 10. Note also that in some embodiments, the TBPS 12 may be configured to be operational on the ground, only.

The TBPS may also include processing circuitry 24. Processing circuitry 24 may include a memory 26 and a processor 28. The processing circuitry may include application specific integrated circuitry instead of or in addition to the processor 28. In particular, in addition to or instead of a processor, such as a central processing unit, and memory, the processing circuitry 24 may comprise integrated circuitry for processing and/or control, e.g., one or more processors and/or processor cores and/or FPGAs (Field Programmable Gate Array) and/or ASICs (Application Specific Integrated Circuitry) adapted to execute instructions. The processor 28 may be configured to access (e.g., write to and/or read from) the memory 26, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory). Thus, the memory 26 may include software that when executed by the processor 28, causes the processor 28 to perform the functions described herein.

In some embodiments, the processor 28 may be configured to implement thermal sensing algorithms 30, facial recognition algorithms 32 and voice generation and speech generation algorithms 34. Such algorithms are commercially available. The thermal sensing algorithms 30 may analyze the thermal reading from the optical and thermal sensing system 16 to determine if the thermal reading exceeds a threshold. The facial recognition algorithms 32 enable the TBPS 12 to identify a passenger. If the identified passenger is determined to be in a febrile condition, the identity and febrile condition (for example, the temperature of the passenger) may be stored in a database format in the memory 26. The memory 26 may also store a thermal image of the passenger and contact tracing information. The voice generation and speech algorithms 34 may be configured to generate audible commands and/or to recognize speech. Thus, in some embodiments, the TBPS 12 may include a microphone and/or a speaker.

Audible commands may include commands: to the passenger on where to stand (“step forward,” for example); to the passenger to present an identification;

to aircraft personnel concerning a state of calibration and/or operation of the TBPS 12; to aircraft personnel concerning tests to be performed by or on the TBPS 12; to aircraft personnel concerning access to stored data and deletion of files; to request identification (such as a username and password) of the aircraft personnel, etc. Audible commands may also include statements such as “You may proceed to board the aircraft,” and “Please move into the designated area for thermal scan.” Note also that the processing circuitry 24 may further be configured to count the number of passengers (or other persons) boarding the aircraft 10.

In some embodiments the Thermo-boarding Pass System 12 may operate in conjunction with, or may include, a graphical user interface (GUI) 36 having at least one input device 38, such as a keyboard, to input information to the TBPS 12. An input device 38 may provide a mode selector 40 to enable an operator of the TBPS 12 to select a mode of operation of the TBPS 12. In some embodiments, the TBPS 12 may have built-in test capabilities. In some embodiments, the GUI 36 enables the user to execute a test and/or maintenance procedure prior to boarding of passengers. Note that the GUI 36 may receive information and transmit information via a Universal Serial Bus (USB) port of the TBPS 12. Also, the GUI 36 may be handheld or part of an instrumentation panel, for example. In some embodiments, the GUI 36 may be outside the aircraft or inside the aircraft. For example, one GUI 36 may be inside the aircraft and another GUI 36 may be outside the aircraft. In this way, an alert can simultaneously or alternatively be provided to flight crew, ground crew, and security personnel. One or more GUIs 36 can be in communication with the TBPS 12 either by electrical conductor, optically, or via RF wireless communications. Note also that in some embodiments, information may be communicated between the TBPS 12 and/or GUIs 36 via a network such as a local area network and/or the Internet.

The mode selector 40 enables the operator to cause the TBPS 12 to operate in one of a plurality of modes. For example, in an aircraft flight servicing mode, the TBPS 12 may be configured to screen flight supply personnel and store the screening information in a first database. In an aircraft maintenance mode, the TBPS 12 may be configured to screen maintenance and cleaning personnel and store the screening information in a second database. In a passenger boarding mode, the TBPS 12 may be configured to screen passengers and store the screening information in a third database. In a flight preparation mode, the TBPS 12 may be configured to screen flight attendants and pilots and store the screening information in a fourth database. In a maintenance mode, the TBPS 12 may be configured to perform built-in tests and tests conducted by an operator of the TBPS 12. In the maintenance mode, the operator may remove or replace the TBPS 12.

The TBPS 12 may be configured to perform a variety of functions including: detecting when an aircraft door is open or closed (as determined by the door relay 22); switching from battery power to aircraft power (and vice versa); performing a thermal scan; identifying a passenger or crew member; count passengers boarding the plane.

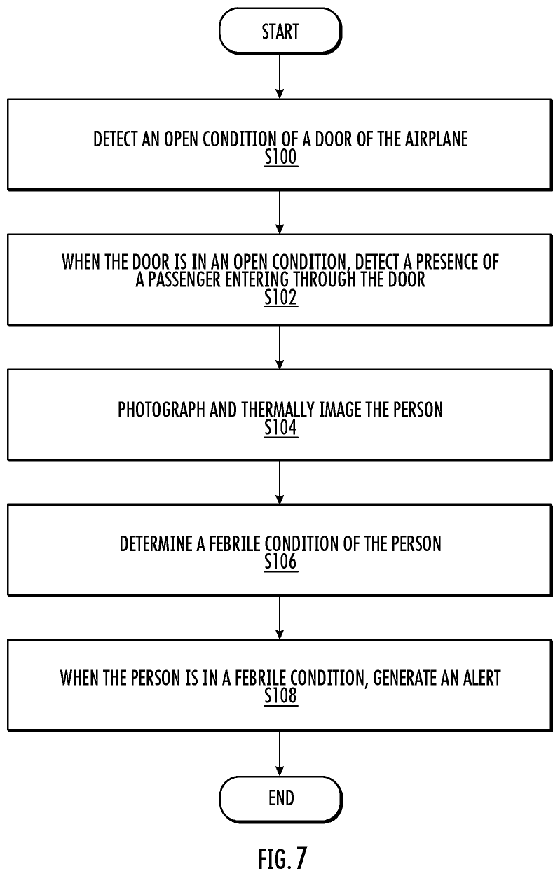

FIG. 7 is a flowchart of an example process for thermal screening and identification of boarding passengers. The process includes detecting an open condition of a door of the aircraft (Block S100). When the door is in an open condition, the process includes detecting a presence of a passenger entering through the door (Block S102). The process also includes photographing and thermally imaging the passenger (Block S104). The process further includes determining a febrile condition of the passenger (Block S106). When the passenger is in a febrile condition, the process includes generating an alert (Block S108). An alert can be a signal to sent to one or more GUIs 36 to cause display of an alert message on the video display 42 and/or emit an audio alert such as an audio frequency tone or sequence of tones or audible voice message that can be heard by those in a near vicinity of the source of the audio alert. For example, if a passenger boarding the aircraft is determined to be in a febrile condition, an audible announcement from a speaker of the GUI 36 that is located in the cabin in the vicinity of the location of the optical and thermal sensing system 16.

In addition to the features described above, some embodiments incorporate or conform to standards and rules set by the Federal Aviation Administration (FAA). For example:

Electrical Loading Requirement: the TBPS 12 installation may be configured to operate at manufacturers operating requirements for the equipment. The installer may ensure through FAA testing that the installation and operation of the TBPS 12 does not cause electrical overload. The installer may also ensure that minimum power requirements are realized.

EWIS (Electrical Wiring Interface Systems) & EZAP (Enhanced Zonal Awareness Procedure Requirement: the TBPS 12 installation should have safe wiring as determined by FAA analysis and inspection.

Thermal Loading Requirement: FAA Testing is required to ensure that the TBPS 12 will not thermally overload from operating in a desired location when also in the proximity of other working equipment.

Wiring Assessment: the installation wire routing should be installed safely and so as not to interfere with other systems nor damage other systems on the aircraft, as determined by FAA inspection.

Flammability Assessment: flammability qualification assessment may be determined by FAA screening non-metallic materials for compliance with the regulations. Those items found to be lacking in qualification may be required to be tested and with test witnessing by the FAA.

Aircraft Safety Assessment: the TBPS components and their installation are required to be analyzed by the FAA for design assurance levels and possible failure modes and affects.

Structural Assessment: TBPS components and installations of the components as well as assembly and installation hardware are reviewed by the FAA for structural strength properties and their adherence to federal regulations and FAA material specifications.

Environmental Qualification: TBPS components and equipment may require aircraft environmental qualification to ensure the equipment as functioning and not functioning is suitable for installation on an aircraft. Environmental qualification is required to be FAA witnessed and approved.

Electromagnetic Interference Qualification: TBPS components after installation must go through a functional FAA witnessed test with all aircraft systems to ensure other aircraft systems and the TBPS do not interfere in the operation of the other.

Human Factors Qualification: the FAA may inspect, qualify, and approve the system for human factors in operation, installation, and functionality.

Aircraft Maintenance Instructions: All components and the installation of the TBPS 12 may be required to have maintenance instructions reviewed and accepted by the FAA. The FAA must also review and approve any airworthiness limitations on the system.

The TBPS 12 may also be configured to conform to other FAA regulations, such as regulations concerning altitude, humidity, shock and crash safety, vibration, magnetic effect, voltage spikes, audio frequency conducted susceptibility, induced signal susceptibility, RF emissions and electrostatic discharge. For example, the TBPS may be electromagnetically shielded to constrain RF emissions and shock-mounted to combat vibration. Other FAA requirements include requirements for structural strength, impact resistance, weight and balance, access for maintenance, servicing and updates, flammability safety, wiring and circuit breakers, interference with other equipment. Conformance to these requirements may be shown by testing before an FAA witness.

According to one aspect, a method is provided for thermal screening of persons. The method includes detecting, via a door relay 22, an open condition of a door 14 of the aircraft. When the door 14 is in an open condition, the method includes detecting, via acoustic or visual detection, a presence of a person entering through the door. The method also includes thermally imaging 16 the person, determining a febrile condition of the person; and when the person is in a febrile condition, alerting aircraft employees.

According to this aspect, in some embodiments, the method includes storing in memory 26 the determination of febrile condition in a database for comparison to subsequent and prior determinations of febrile condition. In some embodiments, the method includes determining, via the facial recognition algorithms 32, an identity of a person in a febrile condition based at least in part on facial recognition of the person. In some embodiments, the method includes storing the identity of the person in a database and correlating, via the processing circuitry 24, the identity of the person with the febrile condition of the person. In some embodiments, the method includes displaying on a video display 42 the febrile condition and identity of the person. In some embodiments, the method includes thermal imaging by a forward looking infrared sensor mounted in a ceiling of the aircraft in proximity of the door 14. In some embodiments, the method includes storing in a first database a febrile condition of an aircraft employee. In some embodiments, the method includes recording, via the processing circuitry 24, a count of persons entering the aircraft that have a febrile condition or that have a non-febrile condition. In some embodiments, the method includes storing thermal imaging data in a database. In some embodiments, the method includes storing contact tracing information for a person determined to be in a febrile condition.

According to another aspect, a thermo-boarding pass system (TBPS) for installation on an aircraft is provided with processing circuitry configured to detect an open condition of a door of the aircraft. When the door is in an open condition, the processing circuitry is configured to: detect a presence of a person entering through the door; thermally and optically image the person; determine a febrile condition of the person; and when the person is in a febrile condition, alert aircraft employees.

According to this aspect, in some embodiments, the processing circuitry is further configured to store the determination of febrile condition in a database for comparison to subsequent and prior determinations of febrile condition. In some embodiments, the processing circuitry is further configured to determine an identity of a person in a febrile condition based on facial recognition of the person. In some embodiments, the processing circuitry is further configured to store the identity of the person in a database and correlate the identity of the person with the febrile condition of the person. In some embodiments, the system is further configured to comply with at least one Federal Aviation Administration (FAA) requirement that includes at least one of an electrical loading requirement, a safe wiring requirement, a thermal overload limit, and a constraint on electromagnetic interference. In some embodiments, the processing circuitry is further configured to process a thermal image captured by a forward looking infrared sensor mounted in a ceiling of the aircraft in proximity of the door. In some embodiments, the processing circuitry is further configured to store in a first database a febrile condition of an aircraft employee. In some embodiments, the processing circuitry is further configured to record a count of persons entering the aircraft that have a febrile condition or that have a non-febrile condition. In some embodiments, the processing circuitry is further configured to store thermal imaging data in a database. In some embodiments, the processing circuitry is further configured to provide storing contact tracing information for a person determined to be in a febrile condition.

As will be appreciated by one of skill in the art, the concepts described herein may be embodied as a method, data processing system, computer program product and/or computer storage media storing an executable computer program. Accordingly, the concepts described herein may take the form of an entirely hardware embodiment, an entirely software embodiment or an embodiment combining software and hardware aspects all generally referred to herein as a “circuit” or “module.” Any process, step, action and/or functionality described herein may be performed by, and/or associated to, a corresponding module, which may be implemented in software and/or firmware and/or hardware. Furthermore, the disclosure may take the form of a computer program product on a tangible computer usable storage medium having computer program code embodied in the medium that can be executed by a computer. Any suitable tangible computer readable medium may be utilized including hard disks, CD-ROMs, electronic storage devices, optical storage devices, or magnetic storage devices.

Some embodiments are described herein with reference to flowchart illustrations and/or block diagrams of methods, systems and computer program products. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer (to thereby create a special purpose computer), special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable memory or storage medium that can direct a computer or other programmable data processing apparatus to function in a particular manner, such that the instructions stored in the computer readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

It is to be understood that the functions/acts noted in the blocks may occur out of the order noted in the operational illustrations. For example, two blocks shown in succession may in fact be executed substantially concurrently or the blocks may sometimes be executed in the reverse order, depending upon the functionality/acts involved. Although some of the diagrams include arrows on communication paths to show a primary direction of communication, it is to be understood that communication may occur in the opposite direction to the depicted arrows.

Computer program code for carrying out operations of the concepts described herein may be written in an object oriented programming language such as Java® or C++. However, the computer program code for carrying out operations of the disclosure may also be written in conventional procedural programming languages, such as the “C” programming language. The program code may execute entirely on the user's computer, partly on the user's computer, as a stand-alone software package, partly on the user's computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider).

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method of screening persons boarding an aircraft to detect a febrile condition of each person, the method comprising:
detecting an open condition of a door of the aircraft;
when the door is in an open condition, detecting a presence of a person entering through the door;
thermally imaging the person;
determining a febrile condition of the person; and
when the person is in a febrile condition, generating an alert.

2. The method of claim 1, further comprising storing the determination of febrile condition in a database for comparison to subsequent and prior determinations of febrile condition.

3. The method of claim 1, further comprising determining an identity of a person in a febrile condition based at least in part on facial recognition of the person.

4. The method of claim 3, further comprising storing the identity of the person in a database and correlating the identity of the person with the febrile condition of the person.

5. The method of claim 1, further comprising displaying on a video display the febrile condition and identity of the person.

6. The method of claim 1, further comprising thermal imaging by a forward looking infrared (FUR) sensor mounted in a ceiling of the aircraft in proximity of the door and directed toward a passenger entering through the door.

7. The method of claim 1, further comprising storing in a first database a febrile condition of an aircraft employee.

8. The method of claim 1, further comprising recording a count of persons entering the aircraft that have a febrile condition or that have a non-febrile condition.

9. The method of claim 1, further comprising storing thermal imaging data in a database.

10. The method of claim 1, further comprising providing storing contact tracing information for a person determined to be in a febrile condition.

11. A thermo-boarding pass system (TBPS) for installation on an aircraft, the system comprising processing circuitry configured to:
detect an open condition of a door of the aircraft;
when the door is in an open condition, detect a presence of a person entering through the door;
thermally image the person;
determine a febrile condition of the person; and
when the person is in a febrile condition, generate an alert.

12. The system of claim 11, wherein the processing circuitry is further configured to store the determination of febrile condition in a database for comparison to subsequent and prior determinations of febrile condition.

13. The system of claim 11, wherein the processing circuitry is further configured to determine an identity of a person in a febrile condition based on facial recognition of the person.

14. The system of claim 13, wherein the processing circuitry is further configured to store the identity of the person in a database and correlate the identity of the person with the febrile condition of the person.

15. The system of claim 11, wherein the system is further configured to comply with at least one Federal Aviation Administration (FAA) requirement that includes at least one of an electrical loading requirement, a safe wiring requirement, a thermal overload limit, and a constraint on electromagnetic interference.

16. The system of claim 11, wherein the processing circuitry is further configured to thermally image a forward looking infrared (FUR) sensor mounted in a ceiling of the aircraft in proximity of the door and directed toward a passenger entering through the door.

17. The system of claim 11, wherein the processing circuitry is further configured to store in a first database a febrile condition of an aircraft employee.

18. The system of claim 11, wherein the processing circuitry is further configured to record a count of persons entering the aircraft that have a febrile condition or that have a non-febrile condition.

19. The system of claim 11, wherein the processing circuitry is further configured to store thermal imaging data in a database.

20. The system of claim 1, wherein the processing circuitry is further configured to provide storing contact tracing information for a person determined to be in a febrile condition.
